(19) United States
(12) Patent Application Publication
(10) Pub. No.: US 2017/0188977 A1
(43) Pub. Date: Jul. 6, 2017

(54) EMOTION ESTIMATION SYSTEM, EMOTION ESTIMATION METHOD, AND COMPUTER-READABLE RECORDING MEDIUM

(71) Applicant: FUJITSU LIMITED, Kawasaki-shi (JP)

(72) Inventor: Teruyuki Sato, Tama (JP)

(73) Assignee: FUJITSU LIMITED, Kawasaki-shi (JP)

(21) Appl. No.: 15/350,903

(22) Filed: Nov. 14, 2016

(30) Foreign Application Priority Data
Jan. 5, 2016 (JP) 2016-000655

(51) Int. Cl. A61B 5/00; A61B 5/024; A61B 5/16

(52) U.S. Cl. CPC A61B 5/7278; A61B 5/165; A61B 5/024; A61B 5/746

(57) ABSTRACT

An emotion estimation system includes a memory; and a processor coupled to the memory, wherein the processor executes a process including, acquiring information on one user's heartbeat intervals measured continuously; classifying user's emotion as any one of at least two types of emotions on the basis of a value indicating a ratio of a value obtained as a result of frequency analysis of the acquired heartbeat interval information to a value indicating a gap between a predicted heartbeat interval calculated on the basis of the acquired heartbeat interval information and an actually obtained heartbeat interval; and performing a different output according to a result of the classifying.

### CROSS-REFERENCE TO RELATED APPLICATION

This application is based upon and claims the benefit of priority of the prior Japanese Patent Application No. 2016-000655, filed on Jan. 5, 2016, the entire contents of which are incorporated herein by reference.

### FIELD

The embodiments discussed herein are related to an emotion estimation system, an emotion estimation method, and a recording medium in which an emotion estimation program is recorded.

### BACKGROUND

In recent years, there has been promoted automation of business with individuals like self-checkout machines and automated teller machines (ATMs). Meanwhile, such automated machines are not always easy to use; for example, when a user pays at the self-checkout, the user may be at a loss to how to operate. At this time, if the user thinks that a factor in preventing the attainment of his/her goal to make payment lies outside him/herself, the user feels an externally-directed aggressive emotion such as irritation. On the other hand, if the user thinks that the factor lies within him/herself, the user feels an emotion such as anxiety or depression.

Furthermore, there has been proposed a technique to use biological information, such as the pulse wave or the heartbeat, to detect one's emotion when he/she operates a machine. For example, there is a proposed method of determining whether a variation in the heart rate is due to a psychological cause or due to exercise and controlling the camera imaging for use in a life log. Furthermore, for example, there is a proposed method of detecting the heartbeat fluctuation to detect the state of player's stress during game play and encourage the player to take a break.

Patent Literature 1: Japanese Laid-open Patent Publication No. 2012-120206

Patent Literature 2: Japanese Laid-open Patent Publication No. 2014-140587

Patent Literature 3: Japanese Laid-open Patent Publication No. 2008-104596

Patent Literature 4: Japanese National Publication of International Patent Application No. 2011-517411

However, even without any exercise, the heart rate varies with the heart rate variability that a living body naturally has; therefore, when a user feels a subjective emotion, it is difficult to determine whether a variation in the heartbeat is the heart rate variability that naturally emerges at rest or due to the emotion.

### SUMMARY

According to an aspect of an embodiment, an emotion estimation system includes a memory; and a processor coupled to the memory, wherein the processor executes a process including, acquiring information on one user's heartbeat intervals measured continuously; classifying user's emotion as any one of at least two types of emotions on the basis of a value indicating a ratio of a value obtained as a result of frequency analysis of the acquired heartbeat interval information to a value indicating a gap between a predicted heartbeat interval calculated on the basis of the acquired heartbeat interval information and an actually obtained heartbeat interval; and performing a different output according to a result of the classifying.

The object and advantages of the invention will be realized and attained by means of the elements and combinations particularly pointed out in the claims.

It is to be understood that both the foregoing general description and the following detailed description are exemplary and explanatory and are not restrictive of the invention, as claimed.

### BRIEF DESCRIPTION OF DRAWINGS

FIG. 1 is a block diagram illustrating an example of a configuration of an emotion estimation system according to a first embodiment;

FIG. 2 is a diagram illustrating an example of emotion assessment;

FIG. 3 is a diagram illustrating an example of heart rate variability data;

FIG. 4 is a diagram illustrating another example of heart rate variability data;

FIG. 5 is a diagram illustrating an example of frequency characteristics of heart rate variability;

FIG. 6 is a diagram illustrating another example of frequency characteristics of heart rate variability;

FIG. 7 is a diagram illustrating an example of first and second ratios;

FIG. 8 is a diagram illustrating another example of emotion assessment;

FIG. 9 is a diagram illustrating another example of the first and second ratios;

FIG. 10 is a diagram illustrating another example of emotion assessment;

FIG. 11 is a flowchart illustrating an example of a determining process according to the first embodiment;

FIG. 12 is a block diagram illustrating an example of a configuration of an emotion estimation system according to a second embodiment;

FIG. 13 is a diagram illustrating an example of frequency characteristics of heart rate variability and LSP;

FIG. 14 is a diagram illustrating another example of frequency characteristics of heart rate variability and LSP;

FIG. 15 is a diagram illustrating still another example of frequency characteristics of heart rate variability and LSP;

FIG. 16 is a flowchart illustrating an example of a determining process according to the second embodiment;

FIG. 17 is a block diagram illustrating an example of a configuration of an emotion estimation system according to a third embodiment;

FIG. 18 is a diagram illustrating an example of log data;

FIG. 19 is a flowchart illustrating an example of a determining process according to the third embodiment; and

FIG. 20 is a diagram illustrating a computer that executes an emotion estimation program.

### DESCRIPTION OF EMBODIMENTS

Preferred embodiments of the present invention will be explained with reference to accompanying drawings. Incidentally, the technology discussed herein is not limited by these embodiments. Furthermore, the embodiments described below can be combined appropriately without causing any contradiction.

### First Embodiment

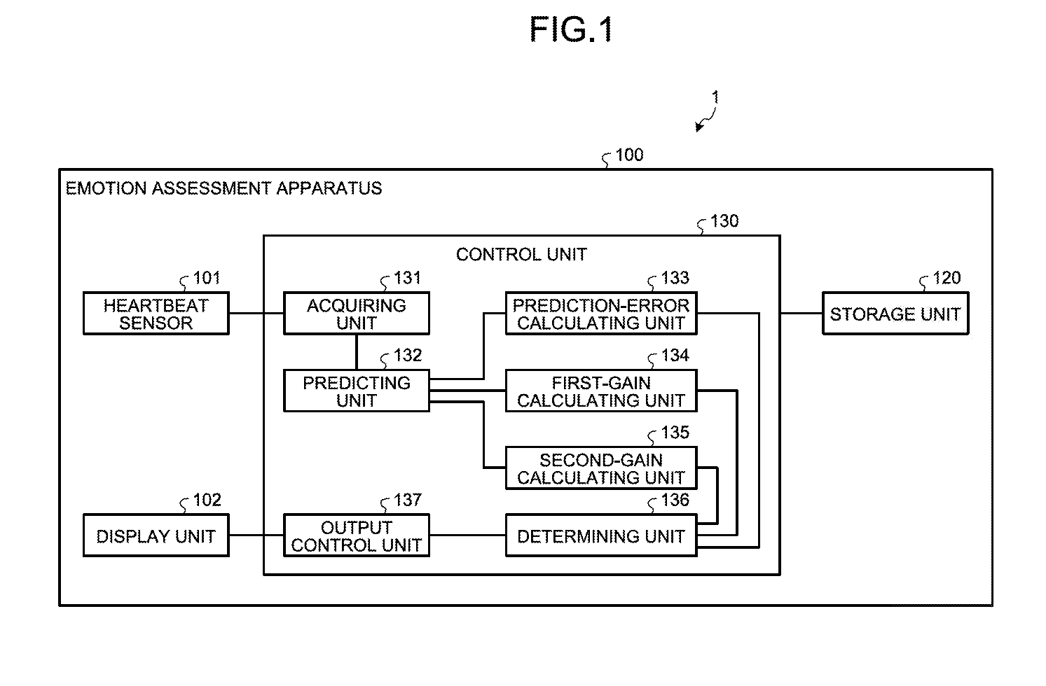

FIG. 1 is a block diagram illustrating an example of a configuration of an emotion estimation system according to a first embodiment. An emotion estimation system 1 illustrated in FIG. 1 includes an emotion assessment apparatus 100. The emotion estimation system 1 can include, for example, predetermined devices, an administrator terminal, a server device, etc. besides the emotion assessment apparatus 100. Incidentally, the predetermined devices include, for example, self-checkout machines, ATMs, etc.

The emotion assessment apparatus 100 acquires information on one user's heartbeat intervals measured continuously. The emotion assessment apparatus 100 classifies user's emotion as any one of at least two types of emotions on the basis of a value indicating the ratio of a value obtained as a result of frequency analysis of the acquired heartbeat interval information to a value indicating a gap between a predicted heartbeat interval calculated on the basis of the acquired heartbeat interval information and an actually obtained heartbeat interval. The emotion assessment apparatus 100 performs a different output according to a result of the classification. Accordingly, the emotion assessment apparatus 100 can perform an output according to the emotional abnormal state.

As illustrated in FIG. 1, the emotion assessment apparatus 100 includes a heartbeat sensor 101, a display unit 102, a storage unit 120, and a control unit 130. Incidentally, besides the functional units illustrated in FIG. 1, the emotion assessment apparatus 100 can include various functional units that a known computer has, such as various input devices, an audio output device, etc.

The heartbeat sensor 101 detects a user's heartbeat signal. For example, the heartbeat sensor 101 acquires a user's heartbeat signal on the basis of the difference in potential between electrodes in contact with the user. Incidentally, the electrodes used by the heartbeat sensor 101 correspond to, for example, chest-belt type electrodes or wristwatch type electrodes embedded in small devices (attached to both hands). The heartbeat sensor 101 continuously measures information on the heartbeat intervals, i.e., heart rate variability data on the basis of detected heartbeat signals. The heartbeat sensor 101 outputs the measured heart rate variability data to the control unit 130. Incidentally, the heart rate variability data is, for example, RRI data that associates a time interval between two adjacent R waves of the heartbeat with detection times of the R waves. Furthermore, the heartbeat sensor 101 can output the heart rate at regular time intervals. In this case, the heart rate is in a relation of 60/RRI.

Moreover, the heartbeat sensor 101 can be configured, for example, to optically measure the blood flow to user's earlobe or the like and acquire the pulse wave. A detecting unit of the heartbeat sensor 101 is an optical type if it acquires the pulse wave; a wristwatch type or wristband type (a reflective type), an ear-clips type (a reflective type, a transmission type), etc. can be used. Furthermore, the heartbeat sensor 101 can acquire the pulse wave, for example, on the basis of infrared reflection from user's face with an infrared camera. Moreover, the heartbeat sensor 101 can acquire the pulse wave, for example, with a millimeter-wave sensor. In these cases, the heartbeat sensor 101 outputs heart rate variability data measured on the basis of the pulse wave to the control unit 130.

Here we explain about heart rate variability data and emotion assessment. First, the principle of heart rate variability and the autonomic balance are explained. According to “Method for assessment of biological effects of projected images on the basis of cross-correlation of physiological parameters,” BME, Vol. 18, No. 1, pp. 8-13, March 2004 (hereinafter, referred to as Non Patent Literature 1) by YOSHIZAWA Makoto et al. of Tohoku University, there are two factors of a variation in the heartbeat. The first factor is the fluctuation of the heartbeat that is caused by variations in hemoglobin due to breathing; the heartbeat varies with a period of less than four seconds. The second factor is due to variations in the blood pressure; the heartbeat varies with a period of about ten seconds (the Mayer wave).

The autonomic nervous system that transmits a fluctuation control signal for controlling the fluctuation of the heartbeat has the following properties: the sympathetic nervous system has the low-pass transfer characteristics of transferring a signal of roughly 0.15 Hz or less, and the parasympathetic nervous system has the all-pass transfer characteristics. That is, in a state where the sympathetic nervous system is predominant, only low-frequency (LF) components appear in fluctuation components. On the other hand, in a state where the parasympathetic nervous system is predominant, both LF components and high-frequency (HF) components appear in fluctuation components. A measuring instrument focused on this principle measures the heartbeat intervals for a given length of time (for two to five minutes), and analyzes frequency components composing variations in the heartbeat, and then measures the balance between sympathetic activity and parasympathetic activity at rest by using the ratio of LF components divided by HF components.

In the present application, the linear prediction method based on an autoregressive (AR) model is used as one of methods used in frequency analysis of fluctuation components. The gist of this method is, first, being able to predict the heart rate variability at rest on the basis of previous series by using linear prediction model. Furthermore, a nonstationary fluctuation component is a deviation from the prediction model, and is identified by a residual error and LF components. The second one is mapping an emotion of a user, i.e., a subject for emotion detection on a plane with nonstationary heartbeat variation components and the stress level as the axes.

Subsequently, spectral analysis of heart rate variability based on the AR model is explained. In spectral analysis using the AR model, a method of looking at LF components and HF components is known. The AR model is expressed in the following Equation (1).

$${x}_{s} = {\sum }_{j = 1}^{M} {a}_{j} {x}_{s - j} + {ɛ}_{s} \qquad ( 1 )$$

Equation (1) represents linear prediction of a current sample Xₛ on the basis of a set of previous M samples Xₛ₋₁, Xₛ₋₂, . . . , X_{s-M}. In Equation (1) , a₁, a₂, . . . , a_{M} denote a weight coefficient; εₛ denotes an observation error, i.e., a residual error and means a deviation from prediction. For example, when a user is at a loss to how to operate a machine, the user is under stress and HF components decrease. At this time, if an emotion of anger or irritation is further added, the user's heartbeat or blood pressure is elevated. To determine the emotion, it can be detected by looking at a “deviation” from the prediction model (the AR model) of heart rate variability at rest.

In the AR model, in the event of a nonstationary heartbeat variation, a residual error is increased. Furthermore, in the AR model, in the event of an increase in the heartbeat or blood pressure, there is a decrease in the correlation between blood pressure variability and heart rate variability, and there is a decrease in LP components. According to Non Patent Literature 1, it is at rest that Mayer waves appear clearly, and on the occurrence of a variation in the heart rate that is independent of the blood pressure or a variation in the blood pressure that is independent of the heart rate, such as on the occurrence of a highly emotional reaction, it is suggested that the relationship between the two in the Mayer wave band is weakened. Incidentally, the Mayer wave band is synonymous with LF. From these facts, a deviation from the AR model is obtained by focusing on a residual error and LF components.

Subsequently, emotion assessment based on spectral analysis using the AR model is explained. In the emotion assessment, a two-axis graph is generated by using two assessment amounts. One of the assessment amounts is the ratio of LF components divided by HF components, which means stress. The other assessment amount is the ratio of LF components divided by a residual error, which is a value of assessment that decreases if there is a nonstationary variation component. LF components indicate whether a blood pressure variation component is periodic or not, and decrease in the event of a sporadic variation in the blood pressure. A residual error indicates whether the linear prediction based on the AR model is true or not, and increases if the prediction is wrong. Therefore, the ratio of LF components divided by a residual error, which is the second assessment amount, indicates that with decreasing LF components or increasing residual error, the value of the ratio becomes smaller and the stationarity becomes reduced. In the graph, the ratio of LF components divided by HF components is plotted on y-axis as the stress level, and the ratio of LF components divided by a residual error is plotted on x-axis as the stationarity.

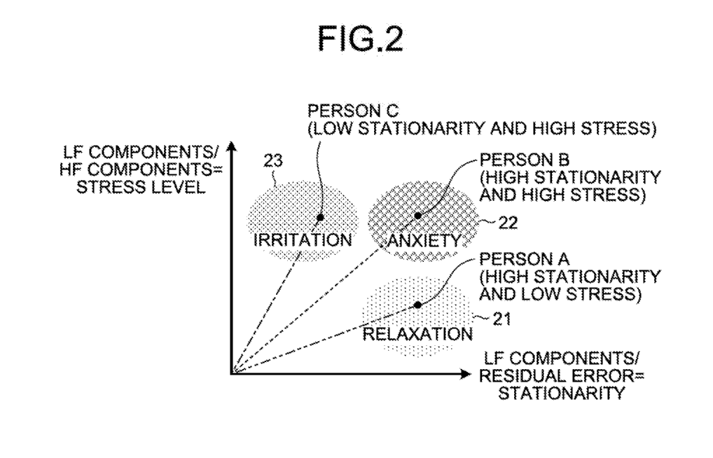

FIG. 2 is a diagram illustrating an example of emotion assessment. In a graph illustrated in FIG. 2, for example, in a case of high stationarity and low stress like a case of Person A, a point on the graph corresponding to Person A exists in an area 21. The area 21 indicates an emotion when one relaxes and is operating a machine. That is, Person A is in a state highly correlated with an emotion of relaxation. Furthermore, in a case of high stationarity and high stress like a case of Person B, a point on the graph corresponding to Person B exists in an area 22. The area 22 indicates an emotion when one feels anxious and is not operating a machine smoothly. That is, Person B is in a state highly correlated with an emotion of anxiety. Moreover, in a case of low stationarity and high stress like a case of Person C, a point on the graph corresponding to Person C exists in an area 23. The area 23 indicates a state where one develops a strong feeling (emotion) of, for example, irritation and, in some cases, is operating a machine in a rough way. That is, Person C is in a state highly correlated with an emotion of irritation. Incidentally, in the present application, a state where one feels an emotion such as anxiety or irritation is referred to as an emotional abnormal state.

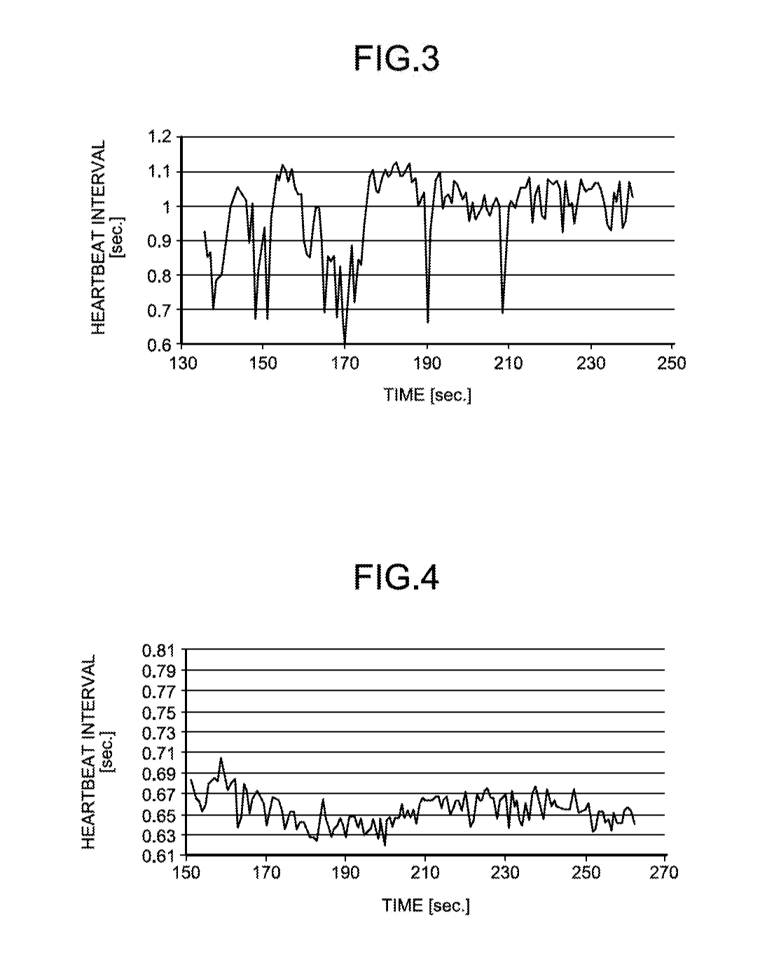

FIG. 3 is a diagram illustrating an example of heart rate variability data. A graph illustrated in FIG. 3 is an example of heart rate variability data in a case where a user is assigned a task. The graph illustrated in FIG. 3 illustrates the heart rate variability data when the user gave an angry expression during the task.

FIG. 4 is a diagram illustrating another example of heart rate variability data. A graph illustrated in FIG. 4 is an example of heart rate variability data of a user who gave an anxious expression during a task. Comparing the graphs illustrated in FIGS. 3 and 4, the graph illustrated in FIG. 4 which is for the user who gave an anxious expression has less variations in the heartbeat. On the other hand, from the graph illustrated in FIG. 3 which is for the user who gave an angry expression, it can be seen that there are stationary heartbeat variations and sporadic heartbeat variations, and the variation width is large.

To return to the explanation of FIG. 1, the display unit 102 is a display device that performs a different output according to a result of classification of user's emotion. The display unit 102 is realized by, for example, an indicator or the like equipped with multiple different color lamps as a display device. The display unit 102 performs a display according to output information received from the control unit 130. Furthermore, the display unit 102 can be realized by, for example, a liquid crystal display or the like as a display device. In this case, the display unit 102 displays thereon results of classification of user's emotion, i.e., information indicating changes in determination result.

The storage unit 120 is realized by, for example, a semiconductor memory device, such as a random access memory (RAM) or a flash memory, or a storage device, such as a hard disk or an optical disk. The storage unit 120 stores therein information used in a process performed by the control unit 130, such as heart rate variability data.

The control unit 130 is realized by, for example, a central processing unit (CPU) or a micro processing unit (MPU) executing a program stored in an internal storage device using a RAM as a work area. Furthermore, the control unit 130 can be realized by an integrated device, such as an application specific integrated circuit (ASIC) or a field programmable gate array (FPGA).

The control unit 130 includes an acquiring unit 131, a predicting unit 132, a prediction-error calculating unit 133, a first-gain calculating unit 134, a second-gain calculating unit 135, a determining unit 136, and an output control unit 137, and realizes or executes the information processing function or action described below. Incidentally, an internal configuration of the control unit 130 is not limited to that illustrated in FIG. 1; the control unit 130 can have any other configuration as long as the control unit 130 is configured to perform information processing described below. Furthermore, the predicting unit 132, the prediction-error calculating unit 133, the first-gain calculating unit 134, the second-gain calculating unit 135, and the determining unit 136 can be integrated into one as a classifying unit. The classifying unit classifies user's emotion as any one of at least two types of emotions on the basis of a value indicating the ratio of a result of frequency analysis of acquired heartbeat interval information to a value indicating a gap between a predicted heartbeat interval calculated on the basis of the acquired heartbeat interval information and an actually obtained heartbeat interval.

The acquiring unit 131 acquires heart rate variability data when the heart rate variability data has been input from the heartbeat sensor 101. That is, the acquiring unit 131 acquires information of one user's heartbeat intervals measured continuously. The acquiring unit 131 outputs the acquired heart rate variability data to the predicting unit 132. Incidentally, heart rate variability data is continuously input from the heartbeat sensor 101, so the acquiring unit 131 also performs the acquisition and output of heart rate variability data continuously. Furthermore, preferably, the acquired heart rate variability data is the one that the acquiring unit 131 has acquired, for example, for about one minute or longer continuously; however, it can be the one that the acquiring unit 131 has acquired, for example, for about 30 seconds or longer continuously. However, if a user is at a loss to how to operate a machine, it seems that the user operates for a reasonably long time; therefore, the acquisition of heart rate variability data continuously for one minute seems to not matter in the operation.

The predicting unit 132 performs frequency analysis using the AR model on heart rate variability data when the heart rate variability data has been input from the acquiring unit 131, and calculates a prediction coefficient for predicting the current heart rate. That is, the predicting unit 132 predicts heart rate variability data on the basis of previous heart rate variability data. Using the above-described Equation (1), the predicting unit 132 performs frequency analysis where a degree to be applied is, for example, a degree of 0 to 16. Incidentally, the predicting unit 132 can obtain a degree by using the Akaike's information criterion (AIC). The predicting unit 132 calculates as many coefficients as the number of the degree as prediction coefficients. The predicting unit 132 outputs a result of the frequency analysis including the prediction coefficient to the prediction-error calculating unit 133, the first-gain calculating unit 134, and the second-gain calculating unit 135. Incidentally, when the predicting unit 132 has received heart rate variability data continuously from the acquiring unit 131, the predicting unit 132 also continuously outputs a result of frequency analysis including a prediction coefficient.

The prediction-error calculating unit 133 calculates a prediction error power in a prediction coefficient when having received a result of frequency analysis from the predicting unit 132. That is, the prediction-error calculating unit 133 calculates the sum of squares Σε² of a residual error in the prediction coefficient. Incidentally, the calculation of a prediction error power can be performed by the predicting unit 132 by using the Levinson-Durbin algorithm along with a prediction coefficient. The prediction-error calculating unit 133 outputs the calculated prediction error power to the determining unit 136. Incidentally, when the prediction-error calculating unit 133 has received a result of frequency analysis continuously from the predicting unit 132, the prediction-error calculating unit 133 also continuously outputs a prediction error power.

The first-gain calculating unit 134 calculates a first gain in a band higher than a first frequency on the basis of a result of frequency analysis when having received the result of frequency analysis from the predicting unit 132. That is, the first-gain calculating unit 134 assesses, out of a transfer function LP(z)=1/(1−Σaⱼz^{-j}) obtained by using the prediction coefficient, |LP(z)|² with the circumference of a unit circle and obtains amplitude characteristics with θ on the abscissa. Incidentally, the circumference of the unit circle is z=e^{iθ}(θ is a normalized angular frequency). The first-gain calculating unit 134 calculates the average of gains in the band higher than the first frequency as a first gain on the basis of the obtained amplitude characteristics. Incidentally, the first frequency is, for example, 0.15 Hz. Incidentally, the first-gain calculating unit 134 can use, for example, a range of 0.15 Hz to 0.4 Hz as the band higher than the first frequency. The first-gain calculating unit 134 outputs the calculated first gain to the determining unit 136. Incidentally, when the first-gain calculating unit 134 has received a result of frequency analysis continuously from the predicting unit 132, the first-gain calculating unit 134 also continuously outputs a first gain.

The second-gain calculating unit 135 calculates a second gain in a band between a second frequency and a third frequency that are a frequency equal to or lower than the first frequency on the basis of a result of frequency analysis when having received the result of frequency analysis from the predicting unit 132. That is, as is the case in the first-gain calculating unit 134, the second-gain calculating unit 135 calculates the average of gains in a band between the second frequency and the third frequency as a second gain on the basis of obtained amplitude characteristics. Incidentally, the second frequency and the third frequency are, for example, 0.15 Hz and 0.05 Hz, respectively. The second-gain calculating unit 135 outputs the calculated second gain to the determining unit 136. Incidentally, when the second-gain calculating unit 135 has received a result of frequency analysis continuously from the predicting unit 132, the second-gain calculating unit 135 also continuously outputs a second gain.

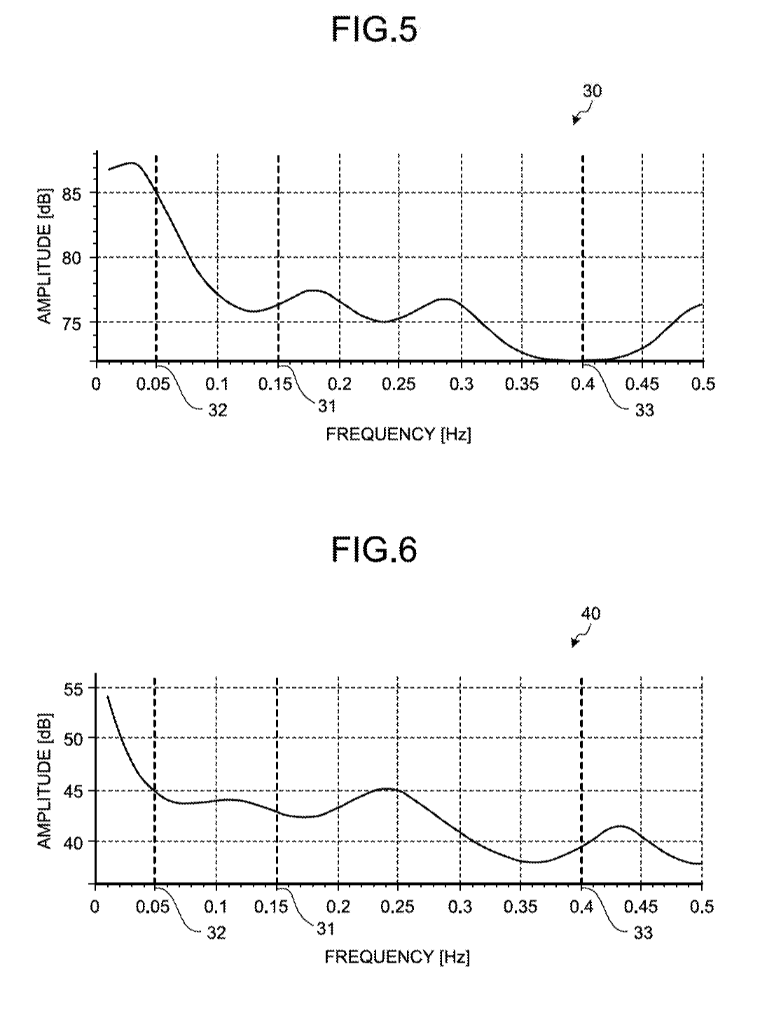

Here, frequency characteristics of heart rate variability is explained with FIGS. 5 and 6. FIG. 5 is a diagram illustrating an example of frequency characteristics of heart rate variability. A graph 30 in FIG. 5 illustrates frequency characteristics of heart rate variability corresponding to the heart rate variability data of the user who gave an angry expression illustrated in FIG. 3. In the graph 30, for example, the first frequency is a frequency 31, the second frequency is the frequency 31, the third frequency is a frequency 32, and an upper limit frequency of the band higher than the first frequency is a frequency 33. The first-gain calculating unit 134 calculates the average of gains in a band from the frequency 31 to the frequency 33 as a first gain. Furthermore, the second-gain calculating unit 135 calculates the average of gains in a band from the frequency 32 to the frequency 31 as a second gain.

FIG. 6 is a diagram illustrating another example of frequency characteristics of heart rate variability. A graph 40 in FIG. 6 illustrates frequency characteristics of heart rate variability corresponding to the heart rate variability data of the user who gave an anxious expression illustrated in FIG. 4. In the graph 40, just like the graph 30, for example, the first frequency is a frequency 31, the second frequency is the frequency 31, the third frequency is a frequency 32, and an upper limit frequency of the band higher than the first frequency is a frequency 33. The first-gain calculating unit 134 calculates the average of gains in a band from the frequency 31 to the frequency 33 as a first gain. Furthermore, the second-gain calculating unit 135 calculates the average of gains in a band from the frequency 32 to the frequency 31 as a second gain.

To return to the explanation of FIG. 1, the determining unit 136 receives a prediction error power, a first gain, and a second gain from the prediction-error calculating unit 133, the first-gain calculating unit 134, and the second-gain calculating unit 135, respectively. The determining unit 136 calculates a first ratio of the second gain to the prediction error power and a second ratio of the second gain to the first gain on the basis of the prediction error power, the first gain, and the second gain. The determining unit 136 plots a point based on the calculated first and second ratios on a graph with the first ratio and the second ratio as the axes, and determines which is the user's emotion out of two or more types of emotions on the basis of an area of the graph where the point has been plotted. That is, the determining unit 136 performs an assessment of the emotion by clustering on a plane with the first ratio and the second ratio as the axes.

Incidentally, when the determining unit 136 has received a prediction error power, a first gain, and a second gain continuously from the prediction-error calculating unit 133, the first-gain calculating unit 134, and the second-gain calculating unit 135, respectively, the determining unit 136 also performs determination of an emotion continuously and outputs a result of the determination continuously.

Specifically, the determining unit 136 determines whether or not the first ratio is equal to or more than a first threshold and the second ratio is less than a second threshold. Incidentally, for example, the first threshold can be set to −60 dB, and the second threshold can be set to −3 dB. When the first ratio is equal to or more than the first threshold and the second ratio is less than the second threshold, the determining unit 136 determines that it is relaxation. In this case, it indicates that the prediction based on the AR model is true and the stress level is low, and the user is in a state of being able to get through a task without being at a loss to how to operate a machine.

When the first ratio is not equal to or more than the first threshold and/or the second ratio is not less than the second threshold, the determining unit 136 determines whether or not the first ratio is equal to or more than the first threshold and the second ratio is equal to or more than the second threshold. When the first ratio is equal to or more than the first threshold and the second ratio is equal to or more than the second threshold, the determining unit 136 determines that it is anxiety. In this case, it indicates that the prediction based on the AR model is true and the stress level is high, and the user does not know what to do and is in a state of anxiety.

When the first ratio is not equal to or more than the first threshold and/or the second ratio is not equal to or more than the second threshold, the determining unit 136 determines whether or not the first ratio is less than the first threshold and the second ratio is equal to or more than the second threshold. When the first ratio is less than the first threshold and the second ratio is equal to or more than the second threshold, the determining unit 136 determines that it is irritation. In this case, it indicates that the prediction based on the AR model is wrong and the stress level is high, and the user is clumsy to use a machine and in a state of irritation.

When the first ratio is not less than the first threshold and/or the second ratio is not equal to or more than the second threshold, the determining unit 136 determines that it is another state. In this case, it indicates that the prediction based on the AR model is wrong and the stress level is low, and it may be a case of aerobic exercise such as walking, however, such an action is unnatural during machine operation, so determination of an emotion is not performed. The determining unit 136 outputs a result of the determination to the output control unit 137. Incidentally, as a result of the determination, for example, a state of irritation corresponds to a first abnormal state, and a state of anxiety corresponds to a second abnormal state. Furthermore, as a result of the determination, for example, another state corresponds to either a normal state or an undeterminable state.

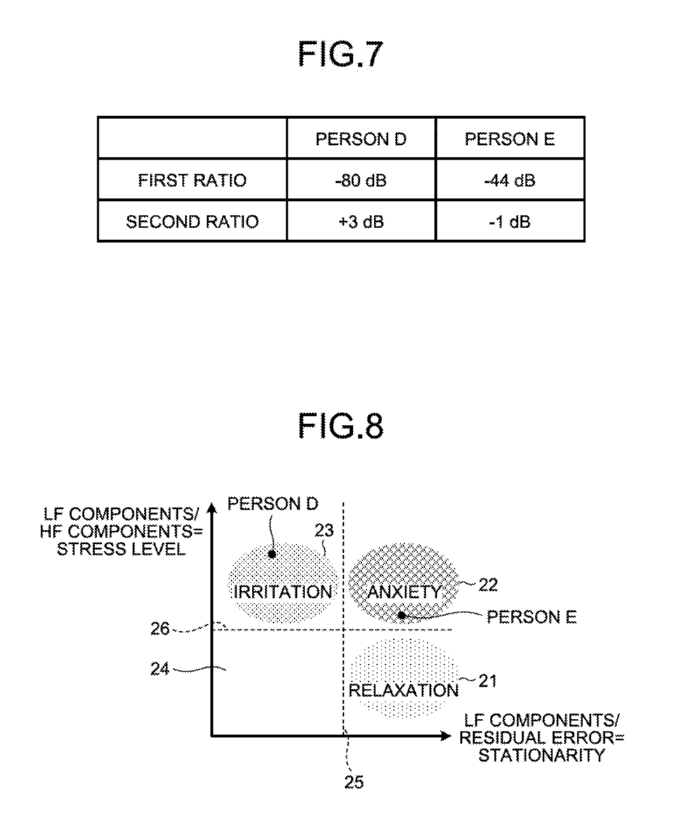

Here, the first and second ratios and emotion assessment are explained with FIGS. 7 to 10. FIG. 7 is a diagram illustrating an example of the first and second ratios. The first and second ratios illustrated in FIG. 7 correspond to the users in FIGS. 5 and 6. Here, the user who gave an angry expression corresponding to the graph 30 in FIG. 5 is Person D, and the user who gave an anxious expression corresponding to the graph 40 in FIG. 6 is Person E. The first ratio of Person D is −80 dB, and the second ratio is +3 dB. Furthermore, the first ratio of Person E is −44 dB, and the second ratio is −1 dB.

FIG. 8 is a diagram illustrating another example of emotion assessment. FIG. 8 illustrates a graph on which the first and second ratios in FIG. 7 are plotted; a point indicating Person D is plotted in the area 23 representing irritation, and a point indicating Person E is plotted in the area 22 representing anxiety. A difference in the second ratio indicating the stress level between Person D and Person E is 4 dB which is a small difference; however, a difference in the first ratio indicating the stationarity is 36 dB which is an obvious difference. Therefore, in the graph of FIG. 8, a first threshold 25 corresponding to the stationarity is set to −60 dB, thereby enabling to distinguish between irritation (anger) and anxiety. Furthermore, in the graph of FIG. 8, a second threshold 26 corresponding to the stress level is set to −3 dB as an anxiety component is smaller than half of a relaxation component, thereby enabling to distinguish between anxiety and relaxation. That is, in the graph of FIG. 8, it is possible to distinguish among three types of emotions: irritation (anger), anxiety, and relaxation. Incidentally, in the graph of FIG. 8, an area 24 corresponds to another state. Furthermore, in the graph of FIG. 8, the areas 21, 22, and 23 are each a portion of an area separated by the first threshold 25 and the second threshold 26; however, the areas 21, 22, and 23 are not limited to this, and can be the whole of an area separated by the first threshold 25 and the second threshold 26.

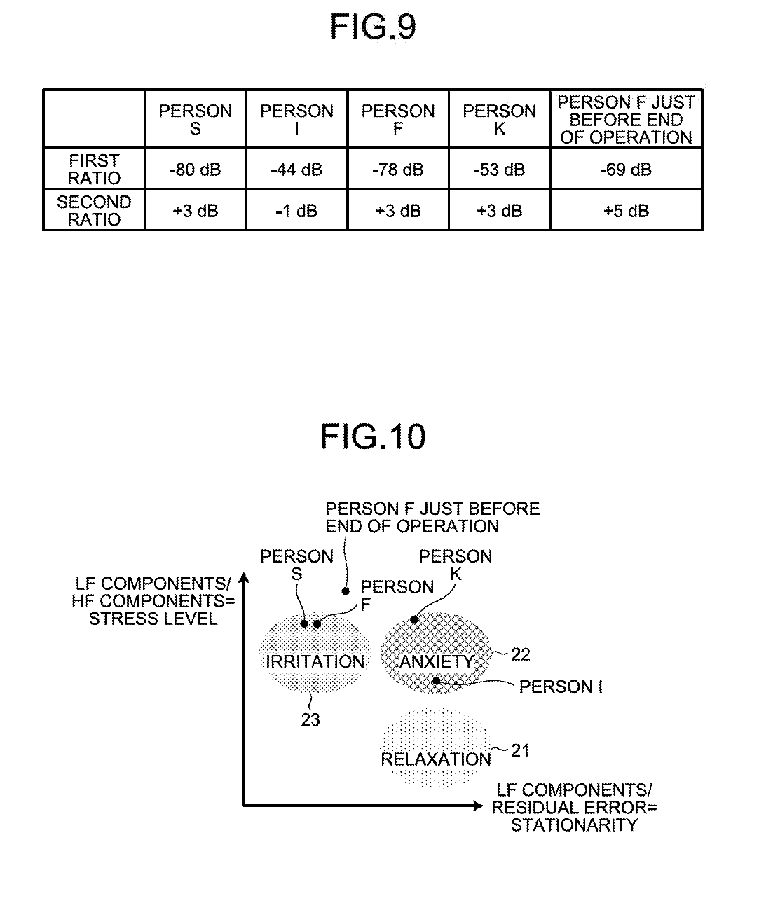

FIG. 9 is a diagram illustrating another example of the first and second ratios. FIG. 9 illustrates, as another example, the first and second ratios of Person S, Person I, Person F, Person K, and Person F just before the end of operation. FIG. 10 is a diagram illustrating another example of emotion assessment. FIG. 10 illustrates a graph on which the first and second ratios in FIG. 9 are plotted; points indicating Person S and Person F are plotted in the area 23 representing irritation, and points indicating Person I and Person K are plotted in the area 22 representing anxiety. Furthermore, a point indicating Person F just before the end of operation is plotted in an area which is higher in the stress level than the area 23.

To return to the explanation of FIG. 1, the output control unit 137 performs a different output according to a result of classification, i.e., a result of determination, when having received the result of determination from the determining unit 136. That is, the output control unit 137 outputs output information for lighting a color lamp according to the result of determination which is, for example, irritation (anger), anxiety, or relaxation to the display unit 102, and causes the display unit 102 to light a color lamp according to the result of determination. In other words, the output control unit 137 outputs an alarm including information that can identify the first or second abnormal state. Furthermore, when it has been determined to be another state, the output control unit 137 does not output an alarm. Incidentally, the alarm can be a display on the display unit 102, or can be the sound of a buzzer or the like (not illustrated).

For example, when the result of determination indicates irritation, the output control unit 137 outputs output information for lighting a red color lamp to the display unit 102. Furthermore, for example, when the result of determination indicates anxiety, the output control unit 137 outputs output information for lighting a yellow color lamp to the display unit 102. Moreover, for example, when the result of determination indicates relaxation, the output control unit 137 outputs output information for lighting a green color lamp to the display unit 102. Furthermore, for example, when the result of determination indicates another state, the output control unit 137 outputs output information for turning a lamp off to the display unit 102. Moreover, the output control unit 137 can output, as output information, for example, information indicating changes in determination result to the display unit 102.

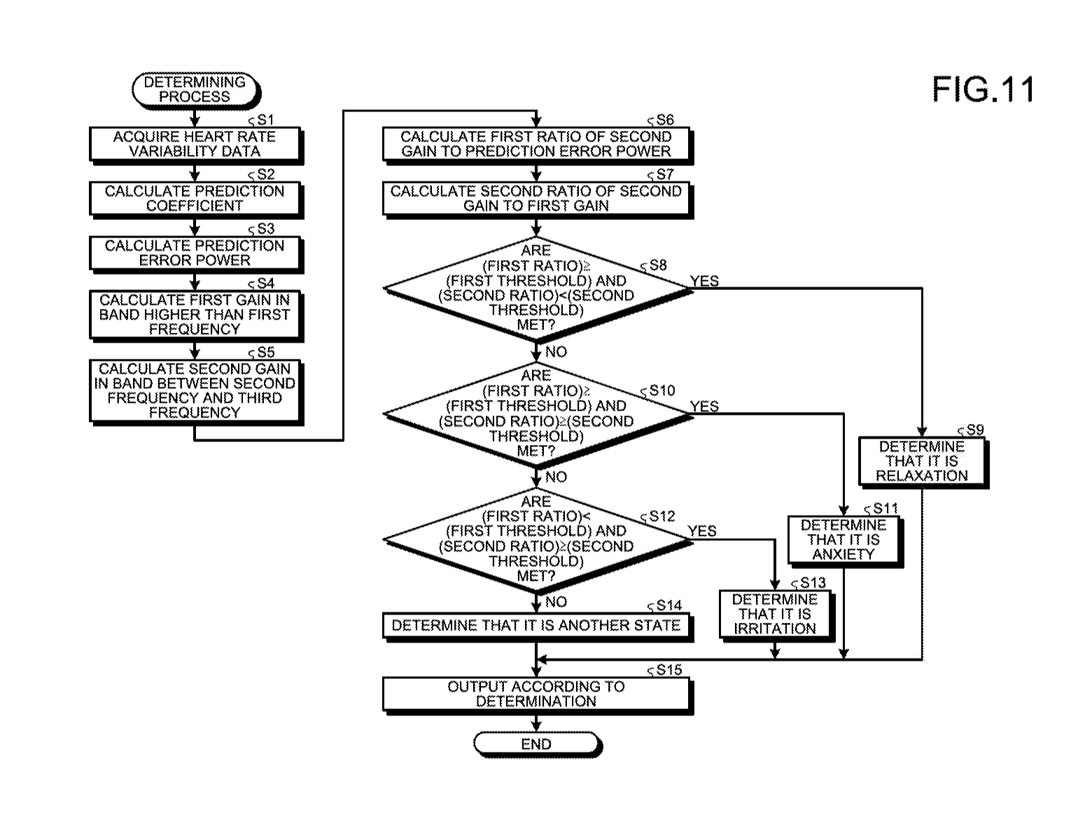

Subsequently, the operation of the emotion estimation system 1 according to the first embodiment is explained. FIG. 11 is a flowchart illustrating an example of a determining process according to the first embodiment.

When heart rate variability data has been input from the heartbeat sensor 101, the acquiring unit 131 of the emotion assessment apparatus 100 acquires the input heart rate variability data (Step S1). The acquiring unit 131 outputs the acquired heart rate variability data to the predicting unit 132.

When having received the heart rate variability data from the acquiring unit 131, the predicting unit 132 performs frequency analysis using the AR model on the received heart rate variability data, and calculates a prediction coefficient for predicting the current heart rate (Step S2). The predicting unit 132 outputs a result of the frequency analysis including the prediction coefficient to the prediction-error calculating unit 133, the first-gain calculating unit 134, and the second-gain calculating unit 135.

When having received the result of the frequency analysis from the predicting unit 132, the prediction-error calculating unit 133 calculates a prediction error power in the prediction coefficient (Step S3). The prediction-error calculating unit 133 outputs the calculated prediction error power to the determining unit 136.

When having received the result of the frequency analysis from the predicting unit 132, the first-gain calculating unit 134 calculates a first gain in a band higher than the first frequency on the basis of the result of the frequency analysis (Step S4). The first-gain calculating unit 134 outputs the calculated first gain to the determining unit 136.

When having received the result of the frequency analysis from the predicting unit 132, the second-gain calculating unit 135 calculates a second gain in a band between the second frequency and the third frequency that are a frequency equal to or lower than the first frequency on the basis of the result of the frequency analysis (Step S5). The second-gain calculating unit 135 outputs the calculated second gain to the determining unit 136.

The prediction error power, the first gain, and the second gain are input to the determining unit 136 from the prediction-error calculating unit 133, the first-gain calculating unit 134, and the second-gain calculating unit 135, respectively. The determining unit 136 calculates a first ratio of the second gain to the prediction error power on the basis of the prediction error power and the second gain (Step S6). Furthermore, the determining unit 136 calculates a second ratio of the second gain to the first gain on the basis of the first gain and the second gain (Step S7).

The determining unit 136 determines whether or not the first ratio is equal to or more than the first threshold and the second ratio is less than the second threshold (Step S8). When the first ratio is equal to or more than the first threshold and the second ratio is less than the second threshold (YES at Step S8), the determining unit 136 determines that it is relaxation (Step S9), and outputs a result of the determination to the output control unit 137.

When the first ratio is not equal to or more than the first threshold and/or the second ratio is not less than the second threshold (NO at Step S8), the determining unit 136 determines whether or not the first ratio is equal to or more than the first threshold and the second ratio is equal to or more than the second threshold (Step S10). When the first ratio is equal to or more than the first threshold and the second ratio is equal to or more than the second threshold (YES at Step S10), the determining unit 136 determines that it is anxiety (Step S11), and outputs a result of the determination to the output control unit 137.

When the first ratio is not equal to or more than the first threshold and/or the second ratio is not equal to or more than the second threshold (NO at Step S10), the determining unit 136 determines whether or not the first ratio is less than the first threshold and the second ratio is equal to or more than the second threshold (Step S12). When the first ratio is less than the first threshold and the second ratio is equal to or more than the second threshold (YES at Step S12), the determining unit 136 determines that it is irritation (Step S13), and outputs a result of the determination to the output control unit 137.

When the first ratio is not less than the first threshold and/or the second ratio is not equal to or more than the second threshold (NO at Step S12), the determining unit 136 determines that it is another state (Step S14), and outputs a result of the determination to the output control unit 137.

When having received the determination result from the determining unit 136, the output control unit 137 outputs output information according to the determination result to the display unit 102 (Step S15). The display unit 102 performs a display according to the output information received from the control unit 130. Accordingly, the emotion assessment apparatus 100 can perform an output according to the emotional abnormal state.

In this way, the emotion assessment apparatus 100 acquires information on one user's heartbeat intervals measured continuously. Furthermore, the emotion assessment apparatus 100 classifies user's emotion as any one of at least two types of emotions on the basis of a value indicating the ratio of a value obtained as a result of frequency analysis of the acquired heartbeat interval information to a value indicating a gap between a predicted heartbeat interval calculated on the basis of the acquired heartbeat interval information and an actually obtained heartbeat interval. Moreover, the emotion assessment apparatus 100 performs a different output according to a result of the classification. Consequently, it is possible to perform an output according to the emotional abnormal state.

Furthermore, the emotion assessment apparatus 100 performs frequency analysis using the AR model on the acquired heartbeat interval information, and calculates a prediction coefficient for predicting the current heart rate. Moreover, the emotion assessment apparatus 100 calculates a prediction error power in the calculated prediction coefficient. Furthermore, the emotion assessment apparatus 100 calculates a second gain in a band lower than a second frequency on the basis of a result of the frequency analysis. Moreover, the emotion assessment apparatus 100 calculates a first ratio of the second gain to the prediction error power, and determines which is the user's emotion out of two or more types of emotions on the basis of a value indicating the calculated first ratio. Furthermore, the emotion assessment apparatus 100 classifies the user's emotion as any one of at least two types of emotions on the basis of the determined emotion. Consequently, it is possible to distinguish between respective emotions of irritation and anxiety.

Moreover, the emotion assessment apparatus 100 calculates, as a second gain, a gain in a band between the second frequency and a third frequency lower than the second frequency. Consequently, it is possible to distinguish between respective emotions of irritation and anxiety.

Furthermore, the emotion assessment apparatus 100 calculates a first gain in a band higher than a first frequency that is equal to or higher than the second frequency on the basis of a result of the frequency analysis. Moreover, the emotion assessment apparatus 100 calculates a second ratio of the second gain to the first gain, and plots a point based on the first and second ratios on a two-dimensional plane with the first and second ratios as the axes, and determines which is the user's emotion out of two or more types of emotions on the basis of an area of the two-dimensional plane where the point has been plotted. Consequently, it is possible to distinguish among respective emotions of irritation, anxiety, and relaxation.

Furthermore, the emotion assessment apparatus 100 performs determination of an emotion continuously, and outputs information indicating changes in determination result. Consequently, it is possible to understand changes in user's emotion.

Moreover, the emotion assessment apparatus 100 determines whether the emotion is a first abnormal state or a second abnormal state. Consequently, it is possible to distinguish between an emotion of irritation and an emotion of anxiety.

Furthermore, the emotion assessment apparatus 100 determines which one is the emotion out of the first abnormal state, the second abnormal state, and another state indicating either a normal state or an undeterminable state. Consequently, it is possible to distinguish between an emotion of irritation and an emotion of anxiety, and also possible to distinguish between the normal state and the undeterminable state.

Moreover, the emotion assessment apparatus 100 outputs an alarm including information that can identify the first or second abnormal state. Consequently, it is possible to distinguish between an emotion of irritation and an emotion of anxiety and inform of the emotion.

Furthermore, when it has been determined to be another state, the emotion assessment apparatus 100 does not output an alarm. Consequently, it is possible to suppress too many information.

### Second Embodiment

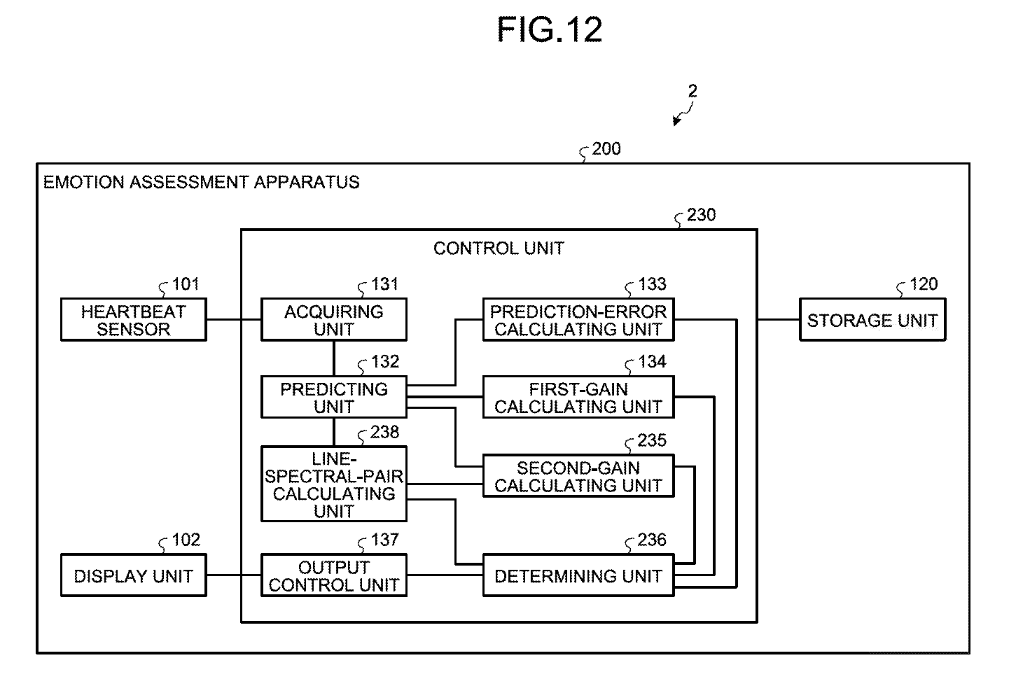

In the above first embodiment, an emotion is determined by using the first ratio of the second gain to the prediction error power and the second ratio of the second gain to the first gain; an emotion can be determined by further using a peak of amplitude characteristics, and an embodiment of this case is explained as a second embodiment. FIG. 12 is a block diagram illustrating an example of a configuration of an emotion estimation system according to the second embodiment. Incidentally, the same component as the emotion estimation system 1 according to the first embodiment is assigned the same reference numeral, thereby description of overlapping configurations and operations is omitted.

A control unit 230 of an emotion assessment apparatus 200 in an emotion estimation system 2 according to the second embodiment further includes a line-spectral-pair calculating unit 238 as compared with the control unit 130 of the emotion assessment apparatus 100 according to the first embodiment. Furthermore, the emotion assessment apparatus 200 includes a second-gain calculating unit 235 and a determining unit 236 instead of the second-gain calculating unit 135 and the determining unit 136 in the first embodiment.

The line-spectral-pair calculating unit 238 receives a result of analysis of an AR model from the predicting unit 132. Incidentally, the predicting unit 132 of the emotion assessment apparatus 200 outputs the analysis result based on the AR model to the line-spectral-pair calculating unit 238. The line-spectral-pair calculating unit 238 calculates line spectral pairs (hereinafter, sometimes referred to as LSP) on the basis of the received analysis result based on the AR model. The line-spectral-pair calculating unit 238 outputs a calculated LSP group to the second-gain calculating unit 235 and the determining unit 236.

Here, how to obtain amplitude characteristics in an LF range and about LSP are explained. When the ratio of LF components divided by HF components is obtained, a method of obtaining the ratio of integrated value of the entire range is known. Here, even if a measurement method of looking at a peak is adopted, there is no problem if a ratio is obtained by applying the same measurement method to both LF components and HF components and then a threshold for the ratio is set appropriately. However, in the present application, one of the axes of a graph is the ratio of LF components divided by a residual error, and the residual error is a scalar value, so an appropriate way to obtain the shape of amplitude characteristics in the LF range becomes a problem. Incidentally, the LF range is a band between the second frequency and the third frequency.

In the second embodiment, as how to obtain amplitude characteristics in the LF range, prediction series a₁, a₂, . . . , a_{M} are converted into a parameter called LSP. Here, LSP is a root of a polynomial of the following Equation (3) composed of the following Equation (2) which is a linear prediction polynomial where the AR model is expressed in Z-transformation.

$$\begin{matrix}A ( z ) = 1 - {\sum }_{k = 1}^{P} {a}_{k} {z}^{- k} & ( 2 ) \\ \{ \begin{matrix}P ( z ) = A ( z ) + {z}^{- ( p + 1 )} A ( {z}^{- 1} ) \\ Q ( z ) = A ( z ) - {z}^{- ( p + 1 )} A ( {z}^{- 1} )\end{matrix} & ( 3 )\end{matrix}$$

We know that the root of Equation (3) is on a unit circle, so what the original root of A(z) is mapped to a pair of two roots on the unit circle is obtained. LSP needs less calculation than numerical analytically resolving a root of A(z), and has a characteristic of being able to grasp frequency characteristics of A(z). This is adopted as a measure of what amplitude characteristics in the LF range are like. More specifically, we know that a tight part of LSP is a peak of frequency characteristics, a thin part is a valley part. That is, the presence or absence of a clear peak can be seen from intervals of LSP in the LF range.

The second-gain calculating unit 235 calculates a second gain on the basis of bands between LSP when having received an LSP group from the line-spectral-pair calculating unit 238. Incidentally, the calculation of a gain can be performed in the same way as the second-gain calculating unit 135 in the first embodiment; however, by calculating the middle point between LSP, the number of plotting of frequency characteristics can be reduced. The second-gain calculating unit 235 outputs the calculated second gain to the determining unit 236.

The determining unit 236 further receives an LSP group from the line-spectral-pair calculating unit 238 as compared with the determining unit 136 in the first embodiment. Using the LSP group, the determining unit 236 assesses whether there is a peak in gain characteristics (frequency characteristics) between the second frequency and the third frequency. That is, the determining unit 236 determines whether there is a peak in the LF range by using an angular frequency obtained by LSP. The assessment is that if the correlation with the blood pressure is lowered by an emotion, no clear peak appears in the LF range. That is, the assessment indicates that there is a peak means there is the stationarity; there is no peak means there is no stationarity. Incidentally, when the determining unit 236 assesses a peak of gain characteristics, the determining unit 236 can assess it by arranging LSP in ascending order of frequency and looking at the minimum length of interval in the second to Nth (N is, for example, 4 to 6) lowest-frequency LSP, i.e., looking at the most tight part of the LSP.

Specifically, the determining unit 236 determines whether or not the first ratio is equal to or more than the first threshold and the second ratio is less than the second threshold. When the first ratio is equal to or more than the first threshold and the second ratio is less than the second threshold, the determining unit 236 determines that it is relaxation.

When the first ratio is not equal to or more than the first threshold and/or the second ratio is not less than the second threshold, the determining unit 236 determines whether or not the first ratio is equal to or more than the first threshold, and the second ratio is equal to or more than the second threshold, and there is a peak in gain characteristics between the second frequency and the third frequency. When all the determination conditions are met, the determining unit 236 determines that it is anxiety. When any of the determination conditions is not met, the determining unit 236 determines whether or not the first ratio is less than the first threshold and the second ratio is equal to or more than the second threshold. When the first ratio is less than the first threshold and the second ratio is equal to or more than the second threshold, the determining unit 236 determines that it is irritation.

When the first ratio is not less than the first threshold and/or the second ratio is not equal to or more than the second threshold, the determining unit 236 determines that it is another state. The determining unit 236 outputs a result of the determination to the output control unit 137. That is, the determining unit 236 determines user's emotion on the basis of whether or not a peak based on intervals of the calculated LSP is in a band between the second frequency and the third frequency.

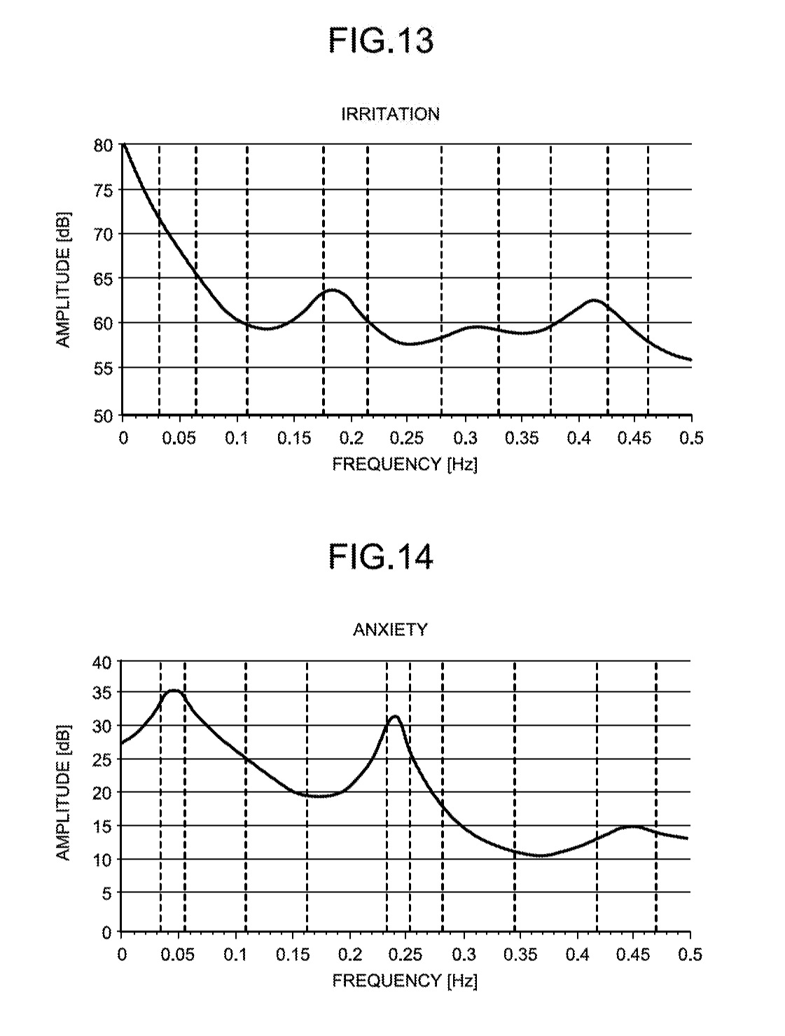

Here, frequency characteristics of heart rate variability and LSP in emotions of irritation, anxiety, and relaxation are explained with FIGS. 13 to 15. Incidentally, in FIGS. 13 to 15, LSP is indicated by a dotted line. FIG. 13 is a diagram illustrating an example of frequency characteristics of heart rate variability and LSP. A graph of FIG. 13 illustrates frequency characteristics of heart rate variability and LSP in a case of an emotion of irritation. In the graph of FIG. 13, no clear peak appears.

FIG. 14 is a diagram illustrating another example of frequency characteristics of heart rate variability and LSP. A graph of FIG. 14 illustrates frequency characteristics of heart rate variability and LSP in a case of an emotion of anxiety. In the graph of FIG. 14, peaks appear in the LF range and the HF range.

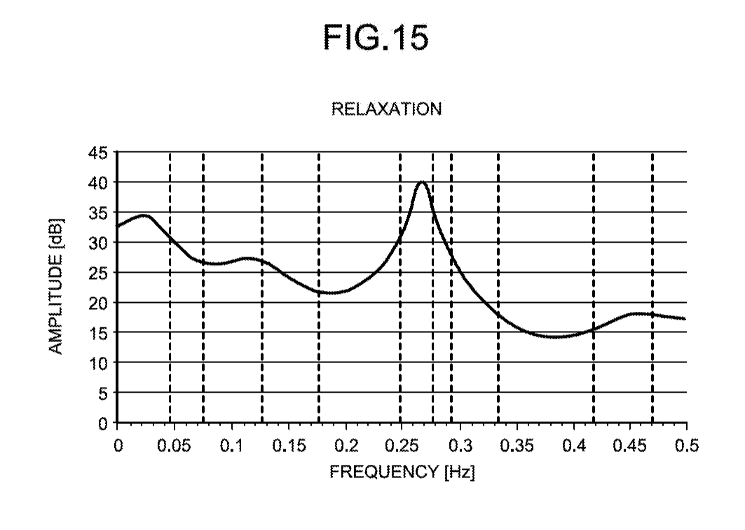

FIG. 15 is a diagram illustrating still another example of frequency characteristics of heart rate variability and LSP. A graph of FIG. 15 illustrates frequency characteristics of heart rate variability and LSP in a case of an emotion of relaxation. In the graph of FIG. 15, a peak appears in the HF range. As illustrated in FIGS. 13 to 15, a tight part of LSP is a peak of frequency characteristics, and a thin part is a part other than the peak. That is, the presence or absence of a clear peak can be seen from intervals of LSP in the LF range in these graphs. In other words, in these graphs, slopes are found by LSP in the LF range and three middle points, and, if signs of the slopes are opposite, there is a peak. On the other hand, if signs of the slopes are the same, there is no peak.

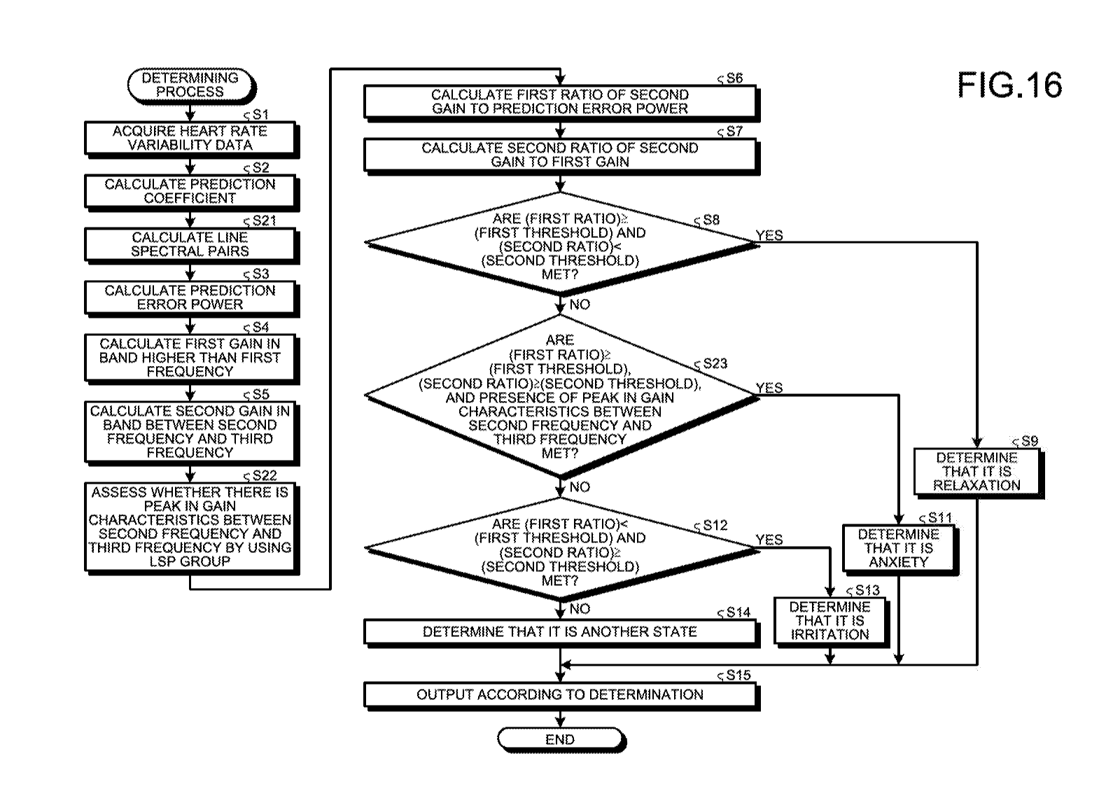

Subsequently, the operation of the emotion estimation system 2 according to the second embodiment is explained. FIG. 16 is a flowchart illustrating an example of a determining process according to the second embodiment. In the following explanation, processes at Steps S1 to S9 and S11 to S15 are the same as the first embodiment, so description of these steps is omitted.

After the process at Step S2, the emotion assessment apparatus 200 performs the following process. An analysis result of an AR model is input to the line-spectral-pair calculating unit 238 from the predicting unit 132. The line-spectral-pair calculating unit 238 calculates line spectral pairs on the basis of the received analysis result based on the AR model (Step S21). The line-spectral-pair calculating unit 238 outputs a calculated LSP group to the second-gain calculating unit 235 and the determining unit 236, and the emotion assessment apparatus 200 goes on to Step S3.

After the process at Step S5, the emotion assessment apparatus 200 performs the following process. Using the LSP group, the determining unit 236 assesses whether there is a peak in gain characteristics between the second frequency and the third frequency (Step S22). Incidentally, a result of the assessment at Step S22 is used in determination at Step S23. The emotion assessment apparatus 200 goes on to Step S6.

After the process at Step S8, the emotion assessment apparatus 200 performs the following process. When the determination at Step S8 is NO, the determining unit 236 determines whether or not the first ratio is equal to or more than the first threshold, and the second ratio is equal to or more than the second threshold, and there is a peak in gain characteristics between the second frequency and the third frequency (Step S23). When all the determination conditions are met (YES at Step S23), the determining unit 236 determines that it is anxiety (Step S11), and the emotion assessment apparatus 200 goes on to Step S15. When any of the determination conditions is not met (NO at Step S23), the emotion assessment apparatus 200 goes on to Step S12. Accordingly, the emotion assessment apparatus 200 can perform an output according to the emotional abnormal state more accurately.

In this way, the emotion assessment apparatus 200 further calculates line spectral pairs on the basis of an analysis result based on the AR model. Furthermore, the emotion assessment apparatus 200 determines user's emotion on the basis of whether there is a peak based on intervals of the calculated LSP in a band between the second frequency and the third frequency. Consequently, it is possible to perform an output according to the emotional abnormal state more accurately.

Moreover, the emotion assessment apparatus 200 calculates a second gain on the basis of bands between the line spectral pairs. Consequently, it is possible to perform an output according to the emotional abnormal state more accurately.

### Third Embodiment

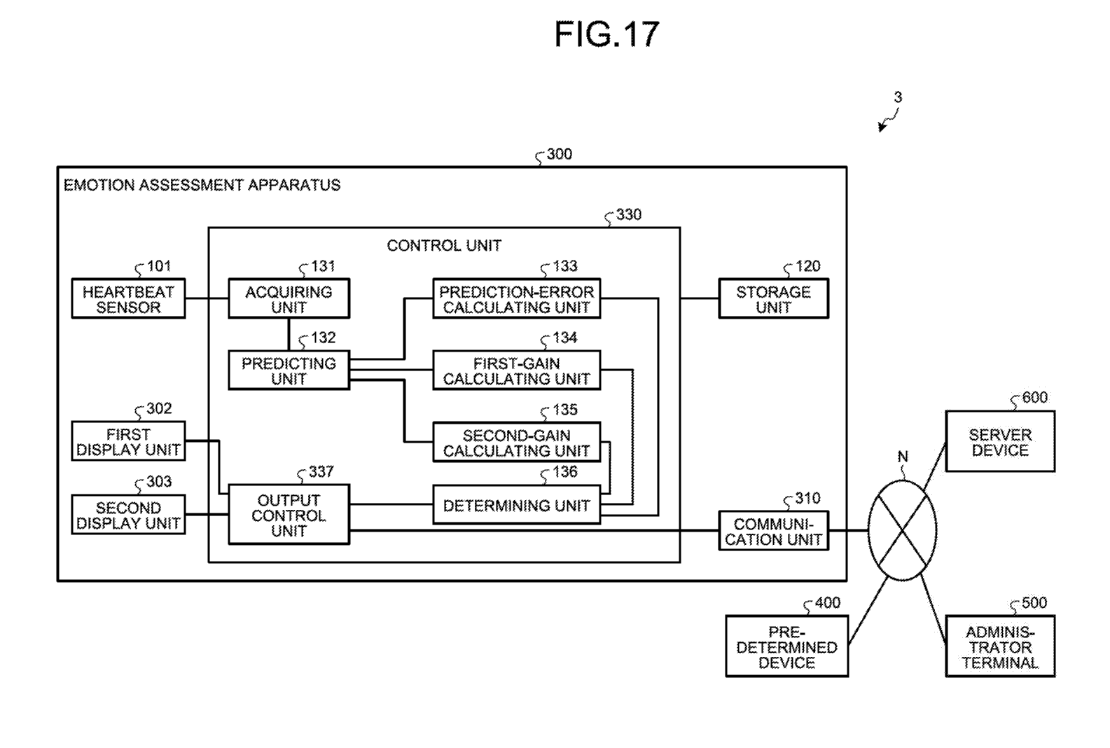

In the above embodiments, output information based on a result of determination is output from the output control unit 137 to the display unit 102; the output information can be further transmitted to a predetermined device that a user is currently operating or an administrator terminal, and an embodiment of this case is explained as a third embodiment. FIG. 17 is a block diagram illustrating an example of a configuration of an emotion estimation system according to the third embodiment. Incidentally, the same component as the emotion estimation system 1 according to the first embodiment is assigned the same reference numeral, thereby description of overlapping configurations and operations is omitted.

An emotion estimation system 3 according to the third embodiment includes an emotion assessment apparatus 300, a predetermined device 400, an administrator terminal 500, and a server device 600. The emotion assessment apparatus 300 includes a first display unit 302 and a second display unit 303 instead of the display unit 102 of the emotion assessment apparatus 100 according to the first embodiment. Furthermore, the emotion assessment apparatus 300 further includes a communication unit 310 as compared with the emotion assessment apparatus 100 according to the first embodiment. Moreover, a control unit 330 of the emotion assessment apparatus 300 includes an output control unit 337 instead of the output control unit 137 of the control unit 130 in the first embodiment.

The first display unit 302 is a display device that performs a different output according to a result of classification of user's emotion like the display unit 102 in the first embodiment. The first display unit 302 is realized by, for example, an indicator or the like equipped with multiple different color lamps as a display device. The first display unit 302 performs a display according to output information received from the control unit 330.

The second display unit 303 is a display device for displaying thereon a variety of information. The second display unit 303 is realized by, for example, a liquid crystal display or the like as a display device. The second display unit 303 displays thereon various screens such as an output screen input from the control unit 330.

The communication unit 310 is realized by, for example, a network interface card (NIC) or the like. The communication unit 310 is connected to the predetermined device 400, the administrator terminal 500, and the server device 600 by wired or wireless via a network N, and is a communication interface that controls communication of information with the predetermined device 400, the administrator terminal 500, and the server device 600. The communication unit 310 transmits output information input from the control unit 330 to the predetermined device 400, the administrator terminal 500, and the server device 600.

The output control unit 337 differs from the output control unit 137 in the first embodiment in that the output control unit 337 further outputs an output screen to the second display unit 303 and transmits output information and log data to the predetermined device 400, the administrator terminal 500, and the server device 600 through the communication unit 310. The output control unit 337 outputs output information for lighting a color lamp according to a result of determination which is, for example, irritation (anger), anxiety, or relaxation to the first display unit 302, and causes the first display unit 302 to light a color lamp according to the determination result. Furthermore, the output control unit 337 transmits output information corresponding to the determination result to the predetermined device 400 and the administrator terminal 500 through the communication unit 310. That is, when the determination result has changed from another state to the first abnormal state or the second abnormal state, or when it is the first abnormal state or the second abnormal state from the start of the determination, the output control unit 337 transmits an alarm to the administrator terminal 500. Incidentally, the alarm is an example of output information.

Moreover, the output control unit 337 generates an output screen for displaying a message according to a determination result input from the determining unit 136, and outputs and displays the generated output screen on the second display unit 303. For example, when the determination result is irritation, the output control unit 337 generates an output screen for displaying a message such as “We apologize for the inconvenience. Our attendant will reach you soon, just a moment, please,” and displays the generated output screen on the second display unit 303. Furthermore, for example, when the determination result is anxiety, the output control unit 337 generates an output screen for displaying a message such as “An expert in operation will reach you soon, just a moment, please,” and displays the generated output screen on the second display unit 303.

The output control unit 337 generates first log data that is records of time-series determination results input from the determining unit 136 together with time stamps. The output control unit 337 transmits the generated first log data to the server device 600 via the communication unit 310 and the network N. Incidentally, the output control unit 337 can use, as timing to transmit the first log data, for example, the timing to transmit the output information to the predetermined device 400 and the administrator terminal 500.

The predetermined device 400 is, for example, a device such as a self-checkout machine or an ATM, and is a device operated by a user. The predetermined device 400 generates second log data that is records of time-series information on user's operations together with time stamps. Incidentally, the information on user's operations is information including information on contents of processing and operation or a screen performed or displayed on the predetermined device 400. When the predetermined device 400 has received output information from the emotion assessment apparatus 300 via the network N, the predetermined device 400 transmits the generated second log data to the server device 600 via the communication unit 310 and the network N.

The administrator terminal 500 is a terminal device used by an administrator who manages the emotion assessment apparatus 300, the predetermined device 400, and the server device 600. For example, when the administrator terminal 500 has received output information from the emotion assessment apparatus 300, the administrator terminal 500 displays the received output information on a display unit (not illustrated). That is, the administrator terminal 500 displays an alarm that is the output information on the display unit (not illustrated). Furthermore, the administrator terminal 500 instructs the server device 600 to perform analysis of the first log data and the second log data via the network N, and receives a result of the analysis and displays the analysis result on the display unit (not illustrated).

The server device 600 is a server device that stores therein first and second log data, performs analysis of the first and second log data on the basis of an instruction to analyze the first and second log data from the administrator terminal 500, and transmits a result of the analysis to the administrator terminal 500. The server device 600 receives the first log data from the emotion assessment apparatus 300 via the network N, and receives the second log data from the predetermined device 400. The server device 600 stores and accumulates the received first and second log data in a storage unit (not illustrated). Furthermore, when having received an instruction to analyze the first and second log data from the administrator terminal 500 via the network N, the server device 600 checks up the accumulated first and second log data, thereby analyzes what is the cause of a change in the emotion. The server device 600 transmits a result of the analysis to the administrator terminal 500 via the network N.

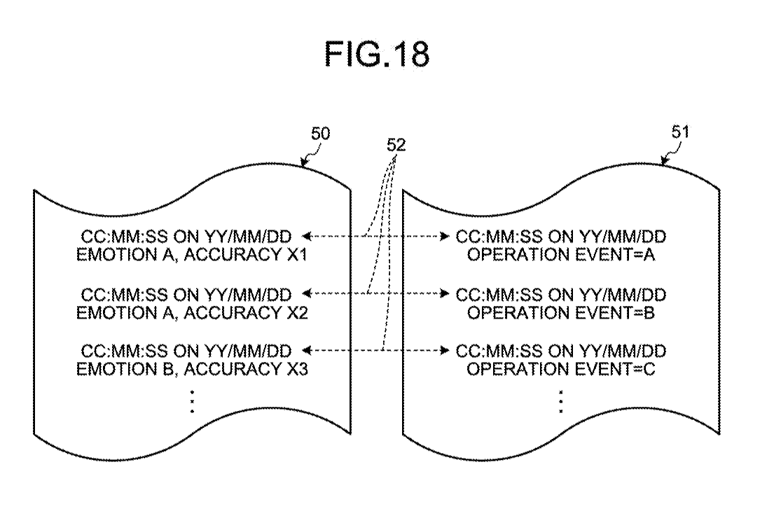

Here, log data is explained with FIG. 18. FIG. 18 is a diagram illustrating an example of log data. As illustrated in FIG. 18, first log data 50 and second log data 51 each store therein results of emotions and operation events with time stamps, i.e., date and time information in an associated manner. The server device 600 checks up the first log data 50 and the second log data 51, and analyzes what is the cause of a change in the emotion. In the example of FIG. 18, through checking up 52, “Emotion A, Accuracy X1” of the first log data 50 is associated with an operation event “A” of the second log data 51. That is, the server device 600 analyzes that the cause of “Emotion A” is the operation event “A”.

Likewise, in the example of FIG. 18, “Emotion A, Accuracy X2” of the first log data 50 is associated with an operation event “B” of the second log data 51. That is, the server device 600 analyzes that the cause of “Emotion A” is the operation event “B”. Furthermore, in the example of FIG. 18, “Emotion B, Accuracy X3” of the first log data 50 is associated with an operation event “C” of the second log data 51. That is, the server device 600 analyzes that the cause of “Emotion B” is the operation event “C”. Incidentally, the accuracy can be set, for example, in such a manner that the higher the accuracy, the closer to the center of each of the areas 21, 22, and 23 in the graph of emotion assessment illustrated in FIG. 8; the lower the accuracy, the closer to the periphery of each of the areas 21, 22, and 23. For example, Accuracies X1 to X3 can be assigned to from the center to the periphery of each area.

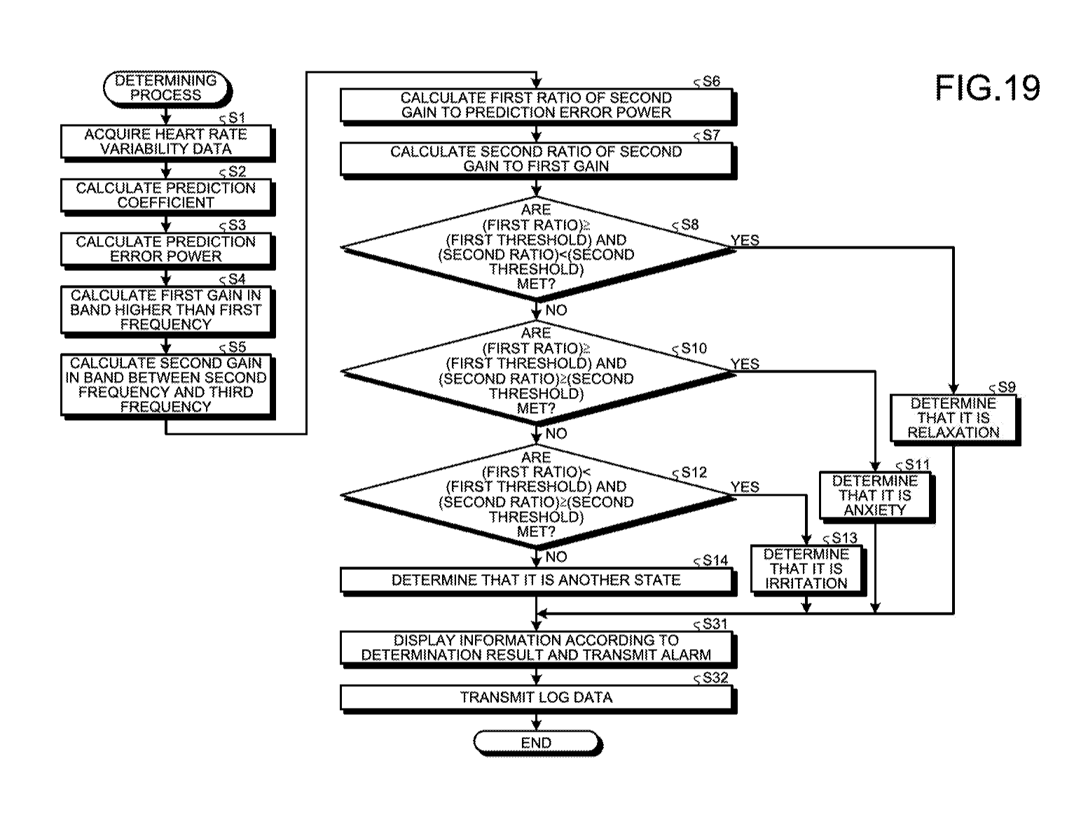

Subsequently, the operation of the emotion estimation system 3 according to the third embodiment is explained. FIG. 19 is a flowchart illustrating an example of a determining process according to the third embodiment. In the following explanation, processes at Steps S1 to S14 are the same as the first embodiment, so description of these steps is omitted.

After the processes at Steps S9, S11, and S13, the emotion assessment apparatus 300 performs the following process. When the output control unit 337 having received a result of determination from the determining unit 136, the output control unit 337 outputs output information according to the determination result to the first display unit 302, and causes the first display unit 302 to light a color lamp according to the determination result. Furthermore, the output control unit 337 generates an output screen for displaying a message according to the determination result input from the determining unit 136, and outputs and displays the generated output screen on the second display unit 303. Moreover, the output control unit 337 transmits output information, i.e., an alarm corresponding to the determination result to the predetermined device 400 and the administrator terminal 500 (Step S31). Incidentally, depending on output information, the predetermined device 400 transmits second log data to the server device 600.

The output control unit 337 generates first log data that is records of time-series determination results input from the determining unit 136 together with time stamps. The output control unit 337 transmits the generated first log data to the server device 600, for example, at the timing to transmit the output information (Step S32). Incidentally, the server device 600 stores and accumulates therein the first log data received from the emotion assessment apparatus 300 and the second log data received from the predetermined device 400. Accordingly, the emotion estimation system 3 can read user's mind and offer a suggestion beforehand, and therefore can suppress user's emotion of anxiety or irritation and contribute to the improvement in customer satisfaction. Furthermore, the emotion estimation system 3 can analyze what kind of operation hurt user's emotion. Moreover, the emotion estimation system 3 can perform the improvement of the predetermined device 400 and the analysis of store operation.

In this way, when a result of determination has changed from another state to the first abnormal state or the second abnormal state, or when it is the first abnormal state or the second abnormal state from the start of the determination, the emotion assessment apparatus 300 transmits an alarm to the administrator terminal 500. Consequently, the administrator can appropriately support a user having an emotion of irritation or anxiety.

Furthermore, in the emotion estimation system 3, the user is a user who is operating the predetermined device 400. Moreover, the predetermined device 400 outputs information on contents of processing and operation or a screen performed or displayed on the predetermined device 400 when the determining unit 136 of the emotion assessment apparatus 300 has determined that the emotion is the first abnormal state or the second abnormal state. Consequently, it is possible to analyze what kind of operation hurt user's emotion.

Incidentally, components of each unit illustrated in the drawings do not necessarily have to be physically configured as illustrated in the drawings. That is, the specific forms of division and integration of components of each unit are not limited to those illustrated in the drawings, and all or some of the components can be configured to be functionally or physically divided or integrated in arbitrary units according to various loads and usage conditions, etc. For example, the predicting unit 132 and the prediction-error calculating unit 133 can be integrated into one unit. Furthermore, the order of the processes illustrated in the drawings is not limited to those illustrated in the drawings; some processes can be performed simultaneously or in different order without causing any contradiction in processing contents.

Moreover, all or any part of processing functions implemented in each apparatus can be executed on a CPU (or a microcomputer such as an MPU or a micro controller unit (MCU)). Furthermore, it goes without saying that all or any part of the processing functions can be executed on a program analyzed and executed by a CPU (or a microcomputer such as an MPU or a micro controller unit (MCU)) or on hardware by wired logic.

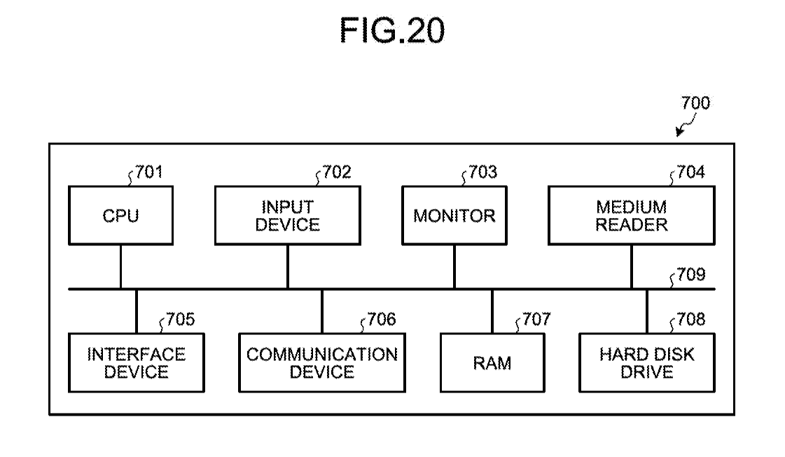

Incidentally, the various processes described in the above embodiments can be realized by causing a computer to execute a program prepared in advance. An example of a computer that executes a program having the same functions as any of the above-described embodiments is explained below. FIG. 20 is a diagram illustrating a computer that executes an emotion estimation program.

As illustrated in FIG. 20, a computer 700 includes a CPU 701 that performs various arithmetic processing, an input device 702 that receives a data input, and a monitor 703. Furthermore, the computer 700 includes a medium reader 704 that reads a program or the like from a storage medium, an interface device 705 for connecting to various devices, and a communication device 706 for connecting to another information processing apparatus by wired or wireless. Moreover, the computer 700 includes a RAM 707 for temporary storage of various information and a hard disk drive 708. These devices 701 to 708 are connected to a bus 709.

An emotion estimation program having the same functions as the acquiring unit 131, the predicting unit 132, the prediction-error calculating unit 133, the first-gain calculating unit 134, the second-gain calculating unit 135, the determining unit 136, and the output control unit 137 illustrated in FIG. 1 is stored in the hard disk drive 708. Furthermore, an emotion estimation program having the same functions as the acquiring unit 131, the predicting unit 132, the prediction-error calculating unit 133, and the first-gain calculating unit 134 illustrated in FIG. 12 can be stored in the hard disk drive 708. Moreover, an emotion estimation program having the same functions as the second-gain calculating unit 235, the determining unit 236, the output control unit 137, and the line-spectral-pair calculating unit 238 illustrated in FIG. 12 can be stored in the hard disk drive 708. Furthermore, an emotion estimation program having the same functions as the acquiring unit 131, the predicting unit 132, the prediction-error calculating unit 133, the first-gain calculating unit 134, the second-gain calculating unit 135, the determining unit 136, and the output control unit 337 illustrated in FIG. 17 can be stored in the hard disk drive 708. Moreover, various data for realizing the emotion estimation program are stored in the hard disk drive 708.

The input device 702 receives, for example, an input of various information such as operation information from an administrator of the computer 700. The monitor 703 has, for example, the same functions as the display unit 102 illustrated in FIG. 1 or 12 or the first and second display units 302 and 303 illustrated in FIG. 17, and performs a display according to output information. The interface device 705 is connected to, for example, the heartbeat sensor 101 illustrated in FIG. 1, 12, or 17. The communication device 706 has, for example, the same functions as the communication unit 310 illustrated in FIG. 17, and is connected to the network N and exchange various information with the predetermined device 400, the administrator terminal 500, and the server device 600.

The CPU 701 reads out programs stored in the hard disk drive 708 and expands the programs into the RAM 707, and executes the programs, thereby performing various processes. These programs can cause the computer 700 to serve as the acquiring unit 131, the predicting unit 132, the prediction-error calculating unit 133, the first-gain calculating unit 134, the second-gain calculating unit 135, the determining unit 136, and the output control unit 137 illustrated in FIG. 1. Furthermore, these programs can cause the computer 700 to serve as the acquiring unit 131, the predicting unit 132, the prediction-error calculating unit 133, the first-gain calculating unit 134, the second-gain calculating unit 235, the determining unit 236, the output control unit 137, and the line-spectral-pair calculating unit 238 illustrated in FIG. 12. Moreover, these programs can cause the computer 700 to serve as the acquiring unit 131, the predicting unit 132, the prediction-error calculating unit 133, the first-gain calculating unit 134, the second-gain calculating unit 135, the determining unit 136, and the output control unit 337 illustrated in FIG. 17.

Incidentally, the above-described emotion estimation program does not necessarily have to be stored in the hard disk drive 708. For example, the computer 700 can read and execute the program stored in a storage medium that the computer 700 can read. The storage medium that the computer 700 can read corresponds to, for example, a portable recording medium, such as a CD-ROM, a DVD, or a universal serial bus (USB) memory, a semiconductor memory such as a flash memory, a hard disk drive, etc. Furthermore, the emotion estimation program can be stored in a device connected to a public circuit, the Internet, a LAN, or the like, so that the computer 700 can read out the emotion estimation program from the device and execute the read program.

It is possible to perform an output according to the emotional abnormal state.

All examples and conditional language recited herein are intended for pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although the embodiments of the present invention have been described in detail, it should be understood that the various changes, substitutions, and alterations could be made hereto without departing from the spirit and scope of the invention.

What is claimed is:

1. An emotion estimation system comprising:
a memory; and
a processor coupled to the memory, wherein the processor executes a process comprising:
acquiring information on one user's heartbeat intervals measured continuously;
classifying user's emotion as any one of at least two types of emotions on the basis of a value indicating a ratio of a value obtained as a result of frequency analysis of the acquired heartbeat interval information to a value indicating a gap between a predicted heartbeat interval calculated on the basis of the acquired heartbeat interval information and an actually obtained heartbeat interval; and
performing a different output according to a result of the classifying.

2. The emotion estimation system according to claim 1, wherein
the classifying includes:
performing frequency analysis using an AR model on the acquired heartbeat interval information, and calculating a prediction coefficient for predicting a current heart rate;
calculating a prediction error power in the calculated prediction coefficient;
calculating a second gain in a band lower than a second frequency on the basis of a result of the frequency analysis; and
calculating a first ratio of the second gain to the prediction error power, and determining which is the user's emotion out of the two or more types of emotions on the basis of a value indicating the calculated first ratio, and
classifying the user's emotion as any one of the at least two types of emotions on the basis of the determined emotion.

3. The emotion estimation system according to claim 2, wherein
the calculating the second gain includes calculating, as the second gain, a gain in a band between the second frequency and a third frequency lower than the second frequency.

4. The emotion estimation system according to claim 2, wherein
the classifying further includes calculating a first gain in a band higher than a first frequency, which is a frequency equal to or higher than the second frequency, on the basis of the result of the frequency analysis, and
the determining further includes calculating a second ratio of the second gain to the first gain, and plotting a point based on the first and second ratios on a two-dimensional plane with the first and second ratios as an axes, and then determining which is the user's emotion out of the two or more types of emotions on the basis of an area of the two-dimensional plane where the point has been plotted.

5. The emotion estimation system according to claim 3, further comprising calculating line spectral pairs on the basis of the analysis result based on the AR model, wherein
the determining includes determining the user's emotion on the basis of whether or not there is a peak based on intervals of the calculated line spectral pairs in the band between the second frequency and the third frequency.

6. The emotion estimation system according to claim 2, wherein
the determining includes determining which one is the emotion out of a first abnormal state, a second abnormal state, and another state indicating either a normal state or an undeterminable state.

7. The emotion estimation system according to claims 6, wherein
the user is a user who is operating a predetermined device, and
the predetermined device outputs information on contents of processing and operation or a screen performed or displayed on the predetermined device when it has been determined at the determining that the emotion is the first abnormal state or the second abnormal state.

8. An emotion estimation method implemented by a computer, the emotion estimation method comprising:
acquiring information on one user's heartbeat intervals measured continuously, using a processor;
classifying user's emotion as any one of at least two types of emotions on the basis of a value indicating a ratio of a value obtained as a result of frequency analysis of the acquired heartbeat interval information to a value indicating a gap between a predicted heartbeat interval calculated on the basis of the acquired heartbeat interval information and an actually obtained heartbeat interval, using a processor.

9. The emotion estimation method according to claim 8, wherein
the classifying includes:
performing frequency analysis using an AR model on the acquired heartbeat interval information, and calculating a prediction coefficient for predicting a current heart rate, using a processor;
calculating a prediction error power in the calculated prediction coefficient, using a processor;
calculating a second gain in a band lower than a second frequency on the basis of a result of the frequency analysis, using a processor;
calculating a first ratio of the second gain to the prediction error power, and determining which is the user's emotion out of the two or more types of emotions on the basis of a value indicating the calculated first ratio, using a processor; and
classifying the user's emotion as any one of the at least two types of emotions on the basis of the determined emotion, using a processor.

10. The emotion estimation method according to claim 9, wherein
the calculating the second gain includes calculating, as the second gain, a gain in a band between the second frequency and a third frequency lower than the second frequency.

11. The emotion estimation method according to claim 9, wherein
the classifying further includes calculating a first gain in a band higher than a first frequency, which is a frequency equal to or higher than the second frequency, on the basis of the result of the frequency analysis, and
the determining further includes calculating a second ratio of the second gain to the first gain, and plotting a point based on the first and second ratios on a two-dimensional plane with the first and second ratios as an axes, and then determining which is the user's emotion out of the two or more types of emotions on the basis of an area of the two-dimensional plane where the point has been plotted.

12. The emotion estimation method according to claim 10, further comprising calculating line spectral pairs on the basis of the analysis result based on the AR model, using a processor, wherein
the determining includes determining the user's emotion on the basis of whether or not there is a peak based on intervals of the calculated line spectral pairs in the band between the second frequency and the third frequency.

13. The emotion estimation method according to claim 9, wherein
the determining includes determining which one is the emotion out of a first abnormal state, a second abnormal state, and another state indicating either a normal state or an undeterminable state.

14. A non-transitory computer-readable recording medium having stored therein an emotion estimation program that causes a computer to execute a process comprising:
acquiring information on one user's heartbeat intervals measured continuously;
classifying user's emotion as any one of at least two types of emotions on the basis of a value indicating a ratio of a value obtained as a result of frequency analysis of the acquired heartbeat interval information to a value indicating a gap between a predicted heartbeat interval calculated on the basis of the acquired heartbeat interval information and an actually obtained heartbeat interval.

15. The non-transitory computer-readable recording medium according to claim 14, wherein
the classifying includes:
performing frequency analysis using an AR model on the acquired heartbeat interval information, and calculating a prediction coefficient for predicting a current heart rate;
calculating a prediction error power in the calculated prediction coefficient;
calculating a second gain in a band lower than a second frequency on the basis of a result of the frequency analysis;
calculating a first ratio of the second gain to the prediction error power, and determining which is the user's emotion out of the two or more types of emotions on the basis of a value indicating the calculated first ratio; and
classifying the user's emotion as any one of the at least two types of emotions on the basis of the determined emotion.

16. The non-transitory computer-readable recording medium according to claim 15, wherein
the calculating the second gain includes calculating, as the second gain, a gain in a band between the second frequency and a third frequency lower than the second frequency.

17. The non-transitory computer-readable recording medium according to claim 15, wherein
the classifying further includes calculating a first gain in a band higher than a first frequency, which is a frequency equal to or higher than the second frequency, on the basis of the result of the frequency analysis, and
the determining further includes calculating a second ratio of the second gain to the first gain, and plotting a point based on the first and second ratios on a two-dimensional plane with the first and second ratios as an axes, and then determining which is the user's emotion out of the two or more types of emotions on the basis of an area of the two-dimensional plane where the point has been plotted.

18. The non-transitory computer-readable recording medium according to claim 16, the process further comprising calculating line spectral pairs on the basis of the analysis result based on the AR model, wherein
the determining includes determining the user's emotion on the basis of whether or not there is a peak based on intervals of the calculated line spectral pairs in the band between the second frequency and the third frequency.

19. The non-transitory computer-readable recording medium according to claim 16, wherein
the determining includes determining which one is the emotion out of a first abnormal state, a second abnormal state, and another state indicating either a normal state or an undeterminable state.
